(19) United States
(12) Patent Application Publication
(10) Pub. No.: US 2024/0092728 A1
(43) Pub. Date: Mar. 21, 2024

(54) HYDROXY-SUBSTITUTED SPHINGOLIPIDS

(71) Applicant: Evonik Operations GmbH, Essen (DE)

(72) Inventors: Xin LU, Essen (DE); Jennifer Schild, Solingen (DE); Sandra Nattland, Essen (DE); Marita Regina Baetsen, Issum (DE); Ursula Maczkiewitz, Essen (DE); Hans Henning Wenk, Muelheim an der Ruhr (DE); Monica Desiree van Logchem, PA Zevenbergen (NL); Lisa Maus, Duisburg (DE)

(73) Assignee: Evonik Operations GmbH, Essen (DE)

(21) Appl. No.: 18/260,420

(22) PCT Filed: Jan. 5, 2022
(86) PCT No.: PCT/EP2022/050107

§ 371 Date: Jul. 5, 2023

(30) Foreign Application Priority Data
Jan. 6, 2021 (EP) 21150361.0

(51) Int. Cl. C07C 235/08; A61K 8/42; A61K 8/49; A61Q 19/08; B01J 31/02; C07C 235/74; C07D 309/10

(52) U.S. Cl. CPC C07C 235/08; A61K 8/42; A61K 8/498; A61Q 19/08; B01J 31/0244; C07C 235/74; C07D 309/10

(57) ABSTRACT

Hydroxy-substituted sphingolipids are useful as constituents in cosmetic formulations. A method for preparing the sphingolipids involves reacting a lysosphingolipid with a hydroxycarboxylic acid or an intramolecular cyclic ester of a hydroxycarboxylic acid. The sphingolipids can be used for therapy or prophylaxis of cell damage, including skin cell damage and cell damage induced by UV radiation.

### FIELD OF THE INVENTION

The invention provides hydroxy-substituted sphingolipids, the preparation and use thereof, and also cosmetic formulations of which they are a constituent.

### PRIOR ART

The purpose of care cosmetics is to maintain the impression of an external youthful appearance, for example that of the skin and hair. There are in principle various ways of achieving this. For example, existing damage to the skin, such as irregular pigmentation or wrinkling, can be corrected by a concealing powder or cream.

Another approach is to protect the skin from environmental influences that result in permanent damage and thus ageing of the skin. The idea is thus to intervene preventively and thereby delay the ageing process.

The most important function of the skin is to protect the body against the uncontrolled escape of water on the one hand while protecting against penetration by harmful chemicals or bacteria and by solar radiation on the other. Long-term exposure of human skin to sunlight can lead to the development of light-induced ageing of the skin and/or pigmentation disorders.

This harmful effect of sunlight is due inter alia to the UVB radiation (280-320 nm) present in the spectrum of sunlight.

There remains an ongoing quest for skin-care substances that are able to reduce or even prevent the development of signs of ageing of the skin caused in particular by solar irradiation.

FR2855049 discloses 6-hydroxy-sphingenin-based ceramides for strengthening of the skin barrier. FR2874610 discloses N-dihydroxyalkyl hydroxyalkanamide derivatives and the use thereof in cosmetics.

The object of the invention was to provide substances that reduce signs of ageing caused by solar irradiation.

### DESCRIPTION OF THE INVENTION

It has surprisingly been found that the ceramide derivatives described hereinbelow are able to achieve the object of the invention and to inhibit the formation of reactive oxygen species (ROS) caused by UV light.

The present invention accordingly provides certain hydroxy-substituted sphingolipids and also the preparation and use thereof.

The invention further provides cosmetic formulations comprising said hydroxy-substituted sphingolipids.

An advantage of the sphingolipids of the invention is the protective effects thereof against DNA damage, in particular UV-induced damage, in the skin in particular.

Another advantage of the sphingolipids of the invention is the thickening effect thereof, particularly in cosmetic formulations.

The present invention thus provides a sphingolipid of the general formula I

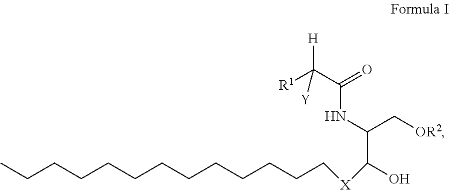

where
R¹ is a hydrocarbon radical having 2 to 54, preferably 2 to 30, more preferably 2 to 18, carbon atoms that is substituted with at least one group selected from —OH and —COOH, and that optionally may be interrupted by at least one —O—,
R² is H, phosphocholine, serine, ethanolamine or a sugar, preferably a sugar or H, more preferably H,
X is CH=CH, CH₂—CH₂ or CH₂—HCOH, preferably CH₂—HCOH, and
Y is selected from —OH and H,

with the proviso that, when R¹ is a linear alkyl radical substituted exclusively with an —OH group in the ω-position, X is CH₂—HCOH.

The sphingolipid of the general formula I has a plurality of stereogenic centres, all of which are covered by the general formula I.

Unless otherwise stated, all stated percentages (%) are percentages by mass.

Preferred sphingolipids of the invention are characterized in that
R¹ is preferably selected from a linear or branched alkyl radical having 2 to 54, preferably 2 to 30, more preferably 2 to 18, carbon atoms that is substituted with at least one group selected from —OH and —COOH, preferably and
R², Y are H, particularly preferably and
X is CH₂—HCOH.

In his preferred embodiment of the invention the alkyl radical of R¹ preferably is straight chained.

Further preferred sphingolipids of the invention are characterized in that
R¹ is preferably selected from a hydrocarbon radical having 2 to 54, preferably 2 to 30, more preferably 2 to 18, carbon atoms that is substituted at the ω-position with at least one group selected from —OH and —COOH, preferably and
R² , Y are H, particularly preferably and
X is CH₂—HCOH.
In his preferred embodiment of the invention the hydrocarbon radical of R¹ preferably is straight chained.

Particularly preferred sphingolipids of the invention are selected from hydroxybutyroyl phytosphingosine

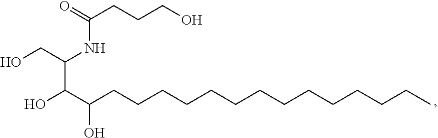 [SMILES: CCCCCCCCCCCCCCC(O)C(O)C(CO)NC(=O)CCCO]

succinoyl phytosphingosine

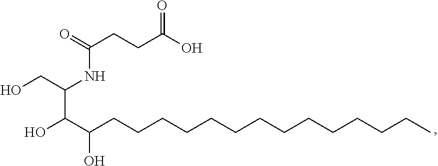 [SMILES: CCCCCCCCCCCCCCC(O)C(O)C(CO)NC(=O)CCC(=O)O]

gluconoyl phytosphingosine

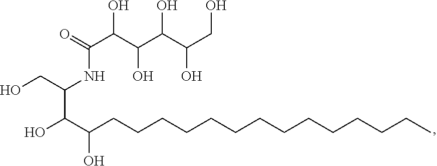 [SMILES: CCCCCCCCCCCCCCC(O)C(O)C(CO)NC(=O)C(O)C(O)C(O)C(O)CO]

lactobionoyl phytosphingosine

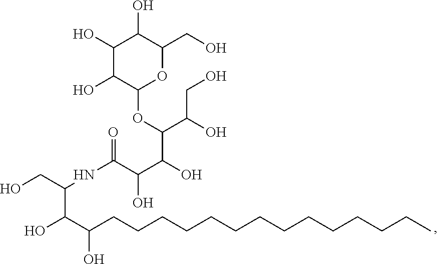 [SMILES: CCCCCCCCCCCCCCC(O)C(O)C(CO)NC(=O)C(O)C(O)C(OC1OC(CO)C(O)C(O)C1O)C(O)CO]

and
2-hydroxy-3,3-dimethyl-hydroxybutyroyl phytosphingosine

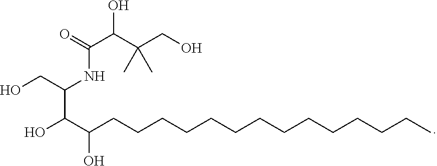 [SMILES: CCCCCCCCCCCCCCC(O)C(O)C(CO)NC(=O)C(O)C(C)(C)CO]

Since the sphingolipids of the invention exhibit an outstanding therapeutic effect, the present invention further provides an inventive sphingolipid for the use in therapy, in particular for the use in prophylaxis, of cell damage, preferably of cell damage induced by UV radiation, in particular skin cell damage, the cell damage in accordance with the invention preferably being DNA damage.

The sphingolipids of the invention can also be employed in purely cosmetic applications, thus the present invention further provides the cosmetic, non-therapeutic use of at least one of the sphingolipids of the invention for the prevention of skin ageing caused by UV radiation.

The sphingolipids of the invention can be readily incorporated into cosmetic formulations. The present invention accordingly further provides a cosmetic formulation comprising at least one sphingolipid of the invention, preferably in an amount of 0.02% by weight to 1.50% by weight, preferably of 0.03% by weight to 1.00% by weight, more preferably of 0.05% by weight to 0.50% by weight, the percentages by weight being based on the total formulation.

The cosmetic formulations of the invention are in particular formulations for sun protection and therefore preferably contain UV light protection filters.

Preferred formulations of the invention are therefore those comprising
at least one sphingolipid of the invention, and
at least one UV light protection filter substance.

The UV light protection filters used may be for example organic substances capable of absorbing ultraviolet radiation and then re-emitting the absorbed energy in the form of longer-wave radiation, for example heat.

UVB filters may be oil-soluble or water-soluble. Examples of oil-soluble UVB light protection filters include:
3-benzylidenecamphor and derivatives thereof, for example 3-(4-methylbenzylidene)camphor,
4-aminobenzoic acid derivatives, for example 2-ethylhexyl 4-(dimethylamino)benzoate and amyl 4-(dimethylamino)benzoate,
esters of cinnamic acid, for example 2-ethylhexyl 4-methoxycinnamate, isopentyl 4- methoxycinnamate, 2-ethylhexyl 2-cyano-3-phenylcinnamate (Octocrylene), esters of salicylic acid, for example 2-ethylhexyl salicylate, 4-isopropylbenzyl salicylate, homomenthyl salicylate,
derivatives of benzophenone, for example 2-hydroxy-4-methoxybenzophenone, 2-hydroxy-4-methoxy-4′-methylbenzophenone, 2,2′-dihydroxy-4-methoxpenzophenone, esters of benzalmalonic acid, for example di-2-ethylhexyl 4-methoxybenzmalonate, triazine derivatives, for example 2,4,6-trianilino-(p-carbo-2′-ethyl-1′-hexyloxy)-1,3,5-triazine, octyl triazone and those described in EP 1180359 and DE 2004/027475, propane-1,3-diones, for example 1-(4-tert-butylphenyI)-3-(4′-methoxphenyl)propane-1,3-dione.

Suitable water-soluble UVB light protection filters include:
2-phenylbenzimidazole-5-sulfonic acid and its alkali metal, alkaline earth metal, ammonium, alkylammonium, alkanolammonium and glucammonium salts,
sulfonic acid derivatives of benzophenone, for example 2-hydroxy-4-methoxybenzophenone-5- sulfonic acid and salts thereof,
sulfonic acid derivatives of 3-benzylidenecamphor, for example 4-(2-oxo-3- bornylidenemethyl)benzenesulfonic acid and 2-methyl-5-(2-oxo-3-bornylidene)sulfonic acid and salts thereof.

Suitable typical UVA light protection filters include in particular derivatives of benzoylmethane, for example 1-(4′-tert-butylphenyI)-3-(4′-methoxphenyl)propane-1,3-dione or 1-phenyl-3-(4′-isopropylphenyl)propane-1,3-dione. It is of course also possible to use mixtures of UV-A and UV-B filters.

In addition to the recited soluble substances, insoluble pigments are also suitable for this purpose, namely finely dispersed metal oxides or salts, for example titanium dioxide, zinc oxide, iron oxide, aluminium oxide, cerium oxide, zirconium oxide, silicates (talc), barium sulfate and zinc stearate. The particles here should have an average diameter of less than 100 nm, e.g. between 5 and 50 nm and in particular between 15 and 30 nm. They may be spherical in shape, although it is also possible to use particles that are ellipsoidal in shape or have a shape that deviates in some other way from spherical. A relatively new class of light protection filters is that of micronized organic pigments, for example 2,2′-methylenebis{6-(2H-benzotriazol-2-yl)-4-(1,1,3,3-tetramethylbutyl)phenol} having a particle size of <200 nm, which is obtainable for example as a 50% aqueous dispersion.

Further suitable UV light protection filters can be found in the review by P. Finkel in SÖFW-Journal 122, 543 (1996).

In association with the formulations of the invention, said formulations preferably comprise lipophilic, hydrophobic UV light protection filter substances, in particular triazine derivatives. Particular preference is given here to using as UV-B filters the UV light protection filter substances 2-ethylhexyl 2-cyano-3-phenylcinnamate, 2,4-bis{[4-(2-ethylhexyloxy)-2-hydroxy]phenyI}-6-(4-methoxyphenyl)-1,3,5-triazine, dioctylbutylamidotriazone, 2- hydroxy-4-methoxybenzophenone, 2-hydroxy-4-methoxy-4′-methylbenzophenone, 2,2′-dihydroxy-4-methoxybenzophenone, di-2-ethylhexyl 4-methoxybenzmalonate, 2,4,6-tris-[anilino-(p-carbo-2′-ethyl-1′-hexyloxy)]-1,3,5-triazine, 2,4-bis[5,1(dimethylpropyl)benzoxazol-2-yl-(4-phenyl)imino]-6-(2-ethylhexyl)imino-1,3,5-triazine, 2,4-bis-{[4-(2-ethylhexyloxy)-2-hydroxy]phenyI}-6-(4-methoxyphenyl)-1,3,5-triazine and 2[4,6-bis(2,4-dimethylphenyl)-1,3, 5-triazin-2-yl]-5-(octyloxy)phenol.

UV-A filters used are preferably 1-(4′-tert-butylphenyI)-3-(4′-methoxphenyl)propane-1,3-dione, 1-phenyl-3-(4′-isopropylphenyl)propane-1,3-dione.

Particularly preferred UV-A filters are 4-(tert-butyl)-4′-methoxydibenzoylmethane (CAS No. 70356-09-1), which is sold by DSM under the Parsol® 1789 brand and by Merck under the trade name Eusolex® 9020, and hydroxybenzophenones in accordance with DE 102004027475, particularly preferably hexyl 2-(4′-diethylamino-2′-hydroxybenzoyl)benzoate (also: aminobenzophenone), available under the name Uvinul A Plus from BASF.

Further preferred UV filter substances are further so-called broadband filters, i.e. filter substances that absorb both UV-A and UV-B radiation. Within this group, preference is given to using 2,2′-methylenebis(6-(2H-benzotriazol-2-yl)-4-(1,1,3,3-tetramethylbutyl)phenol), which is available under the trade name Tinosorb® M from Ciba Chemikalien GmbH, and 2-(2H-benzotriazol-2-yl)-4-methyl-6[2-methyl-3[1,3,3,3-tetramethyl-1-[(trimethylsilyl)oxy]disiloxanyl]propyl]phenol (CAS No.: 155633-54-8), which has the INCI name Drometrizole Trisiloxane.

In addition to the two groups of primary UV light protection filters mentioned above, it is also possible to employ secondary light stabilizers of the antioxidant type that arrest the photochemical reaction chain initiated when UV radiation penetrates the skin.

The cosmetic formulations of the invention comprise UV light protection filters preferably in an amount of 0.01% by weight to 15% by weight, preferably 0.05% by weight to 10% by weight, more preferably 0.1% by weight to 5% by weight, based on the overall formulation.

Preferably, the cosmetic formulations of the invention comprise a combination of two or more different UV light protection filters.

When both UVA and UVB light protection filters are used in a formulation of the invention, the weight ratio of these filters is preferably 1:2 to 1:4.

The formulations of the invention can further comprise at least one additional component selected from the following group:
emollients,
emulsifiers,
thickeners/viscosity regulators/stabilizers,
antioxidants,
hydrotropes (or polyols),
solids and fillers,
film formers,
pearlescence additives,
deodorant and antiperspirant active substances,
insect repellents,
self-tanning agents,
preservatives,
conditioning agents,
perfumes,
dyes,
odour absorbers,
cosmetic active substances,
care additives,
superfatting agents,
solvents.

Substances that can be used as exemplary representatives of the individual groups are known to those skilled in the art and can be found for example in German patent application DE 102008001788.4. This patent application is hereby incorporated as reference and thus forms part of the disclosure.

As regards further optional components and the employed amounts of these components, express reference is made to the relevant handbooks known to those skilled in the art, for example K. Schrader, “Grundlagen and Rezepturen der Kosmetika” [Fundamentals and formulations of cosmetics], 2nd edition, pages 329 to 341, Hüthig Buch Verlag Heidelberg. The amounts of each additive are guided by the intended use.

Typical frame formulations for particular applications are known prior art and are contained for example in the brochures of the manufacturers of the particular base materials and active ingredients. These existing formulations can generally be adopted unchanged. However, when necessary for adjustment and optimization, the desired modifications can be executed in a straightforward manner through simple tests.

The present invention further provides a method for preparing sphingolipids, in particular for preparing the sphingolipids of the invention, comprising the method steps of
I) providing a first component, at least a lysosphingolipid of the general formula II

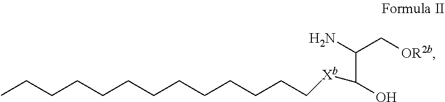

where
R²b is H, phosphocholine, serine, ethanolamine or a sugar, preferably a sugar or H, more preferably H, and X^{b} is CH═CH, CH₂-CH₂ or CH₂-HCOH, preferably CH₂-HCOH, and
II) providing a second component, at least an intramolecular cyclic ester of a hydroxycarboxylic acid of the formula R^{1b}CY^{b}HCOOH,
where
R^{1b} is a hydrocarbon radical having 2 to 54, preferably 2 to 30, more preferably 2 to 18, carbon atoms that is substituted with at least one group selected from —OH and —COON, and that optionally may be interrupted by at least one —O—,
Y^{b} is selected from —OH and H, and
III) reacting the first with the second component to obtain the sphingolipid, and optionally
IV) purifying the sphingolipid.

The method of the invention is preferably characterized in that method step III) is carried out within a temperature range from 40° C. to 95° C., preferably from 50° C. to 80° C., more preferably from 60° C. to 70° C.

The present invention further provides an alternative method for preparing sphingolipids, in particular for preparing the sphingolipids of the invention, comprising the method steps of
A) providing a first component, at least a lysosphingolipid of the general formula II

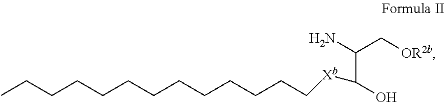

where
R^{2b} is H, phosphocholine, serine, ethanolamine or a sugar, preferably a sugar or H, more preferably H, and
X^{b} is CH═CH, CH₂-CH₂ or CH₂-HCOH, preferably CH2-HCOH, and
B) providing a second component, at least a hydroxycarboxylic acid of the formula R^{1b}CY^{b}HCOOH
where
R^{1b} is a hydrocarbon radical having 2 to 54, preferably 2 to 30, more preferably 2 to 18, carbon atoms that is substituted with at least one group selected from —OH and —COON, and that optionally may be interrupted by at least one —O—,
Y^{b} is selected from —OH and H, and
C) reacting the first with the second component, using at least one coupling reagent for activation of the hydroxycarboxylic acid, to obtain the sphingolipid, and optionally
D) purifying the sphingolipid.

The alternative method of the invention is preferably characterized in that in method step C) the coupling reagent used is at least one selected from the group comprising, preferably consisting of, dicyclohexylcarbodiimide, diisopropylcarbodiimide, 1-(3-dimethylaminopropyl)-3-ethylcarbodiimide hydrochloride, N-cyclohexyl-N′-(2′-morpholinoethyl)carbodiimide metho-p-toluenesulfonate, N-benzyl-N′-3′ dimethylaminopropylcarbodiimide hydrochloride, 1-ethyl-3-(3-dimethylaminopropyl)carbodiimide, N-ethylcarbodiimide hydrochloride and carbonyldiimidazole, particularly preferably dicyclohexylcarbodiimide and diisopropylcarbodiimide.

The alternative method of the invention is preferably characterized in that in method step C) at least one catalyst selected from the group comprising, preferably consisting of, N-ethyldiisopropylamine, trialkylamines, pyridine, 4-dimethylaminopyridine and hydroxybenzotriazole, in particular hydroxybenzotriazole, is used.

The method of the invention is preferably characterized in that method step C) is carried out within a temperature range from 40° C. to 95° C., preferably from 50° C. to 80° C., more preferably from 55° C. to 65° C.,

Methods preferred according to the invention preferably result in the sphingolipids described above as preferred according to the invention.

The examples that follow describe the present invention by way of example, without any intention that the invention, the scope of application of which is apparent from the entirety of the description and the claims, be restricted to the embodiments specified in the examples.

The Following Figures Form Part of the Examples:

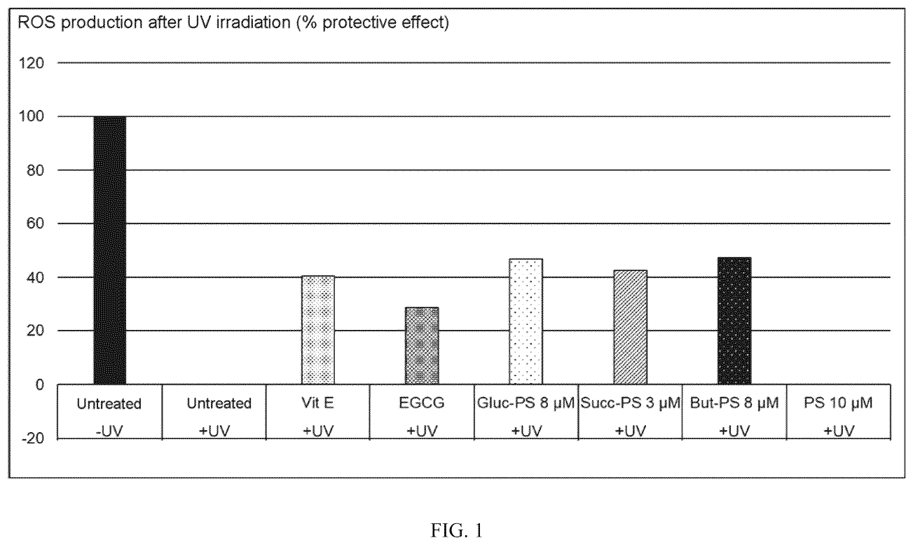

FIG. 1: ROS production after UV irradiation

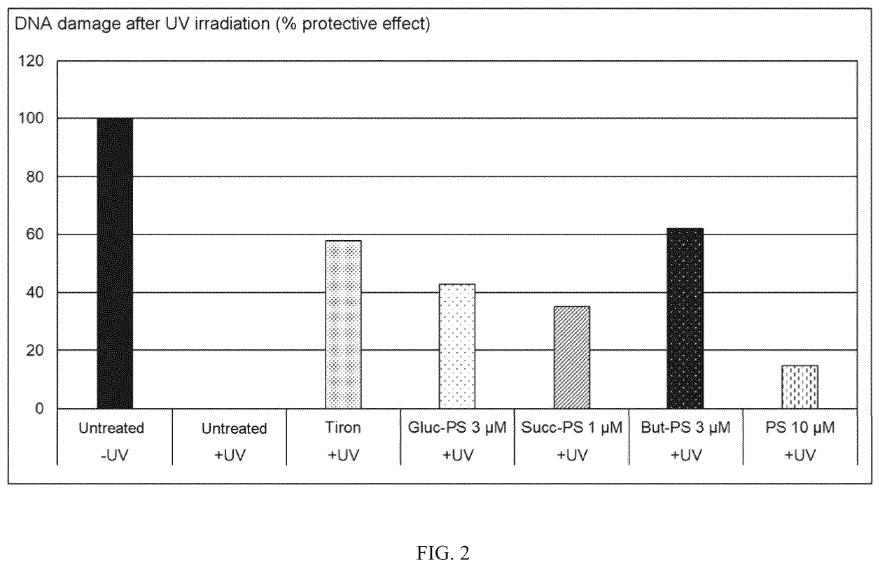

FIG. 2: DNA damage after UV irradiation

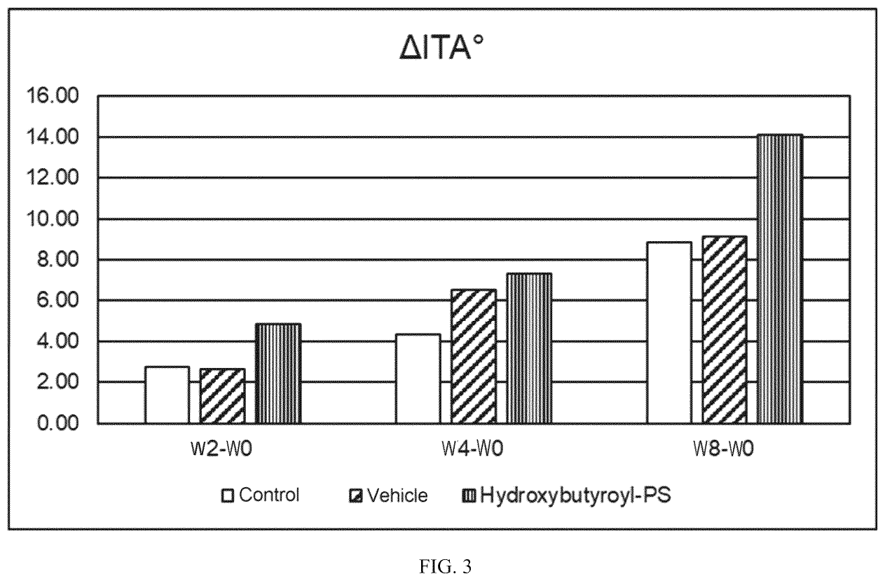

FIG. 3: Increase in ITA° after application of the test formulations for two, four and eight weeks

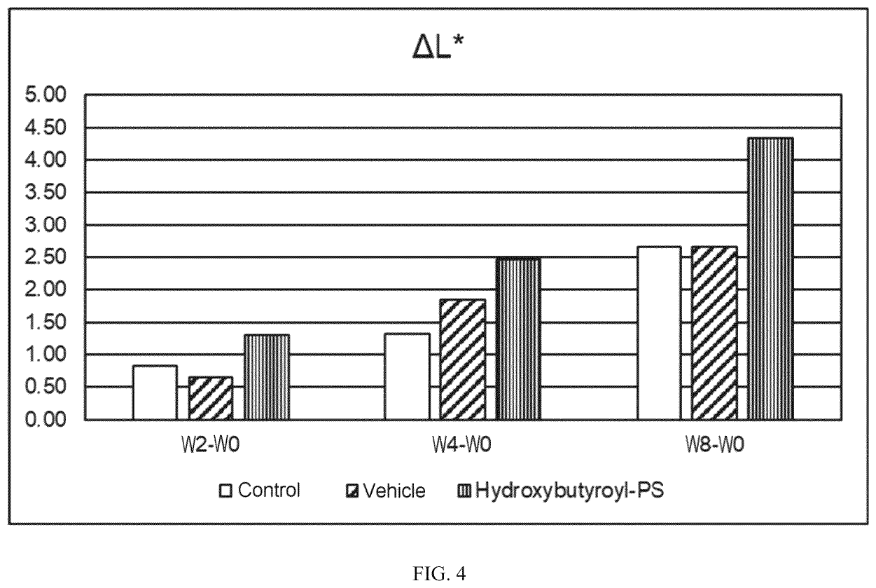

FIG. 4: Increase in L* after application of the test formulations for two, four and eight weeks

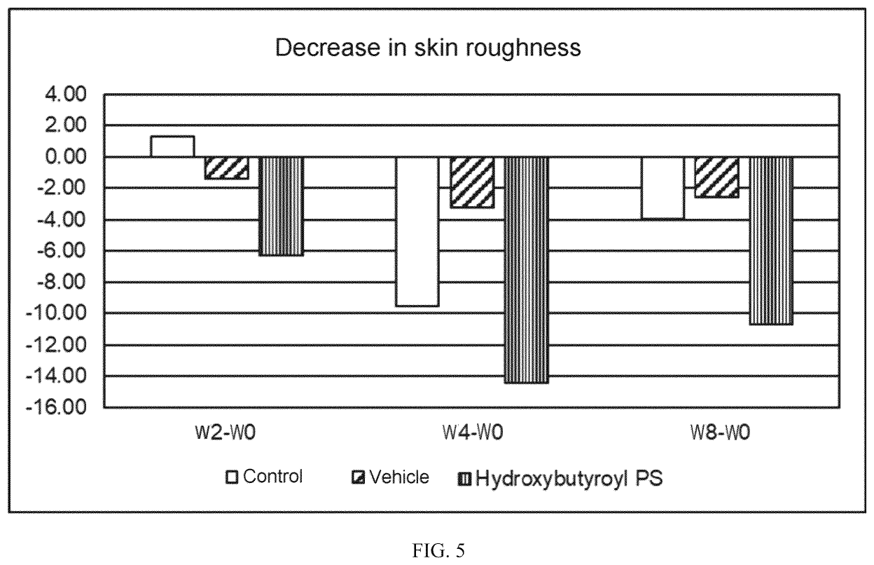

FIG. 5: Decrease in skin roughness after application of the test formulations for two, four and eight weeks

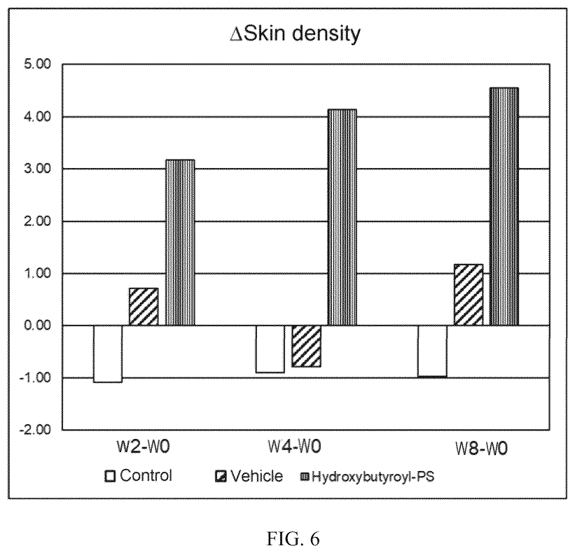

FIG. 6: Increase in skin density after application of the test formulations for two, four and eight weeks

### EXAMPLES

### Example 1: 4-Hydroxybutyroyl phytosphingosine

Phytosphingosine and γ-butyrolactone are dissolved in methanol in a 1:1 molar ratio. The mixture is reacted at 65° C. until phytosphingosine can no longer be detected. Cooling to room temperature affords crystals of 4-hydroxybutyroyl phytosphingosine, which were filtered off with suction and washed with a 1:1 THF/water mixture. The yield is >80% and the purity is >90%.

Example 1b: 6-Hydroxyhexanoyl phytosphingosine

Phytosphingosine and ε-caprolactone are dissolved in methanol in a 1:1 molar ratio. The mixture is reacted at 65° C. until phytosphingosine can no longer be detected. Cooling to room temperature affords crystals of 6-hydroxyhexanoyl phytosphingosine, which were filtered off with suction and washed with a 1:1 THF/water mixture. The yield is >80% and the purity is >90%.

### Example 1c (Not According to the Invention): 3-Hydroxypropioyl phytosphingosine

Phytosphingosine and β-propiolactone are dissolved in methanol in a 1:1 molar ratio. The mixture is reacted at 65° C. until phytosphingosine can no longer be detected. Cooling to room temperature affords crystals of 3-hydroxypropioyl phytosphingosine, which were filtered off with suction and washed with a 1:1 THF/water mixture. The yield is >80% and the purity is >90%.

### Example 2: Succinoyl phytosphingosine

Phytosphingosine and succinic anhydride are dissolved in methanol in a 1:1 molar ratio. The mixture is reacted at 65° C. until phytosphingosine is no longer detected. Cooling to room temperature affords crystals of Succinoyl phytosphingosine, which were filtered off with suction and washed with a 1:1 THF/water mixture. The yield is >80% and the purity is >90%.

### Example 3: Gluconoyl phytosphingosine

Phytosphingosine and d-gluconolactone are dissolved in methanol in a 1:1 molar ratio. The mixture is reacted at 65° C. until phytosphingosine can no longer be detected.

Workup is by crystallization with a gradual lowering of the temperature to 10° C.

The product is filtered off with the aid of vacuum and washed with ethanol. After drying to constant mass, the yield is >80% and the purity is >90%.

### Example 4: Lactobionoyl phytosphingosine

71.66 g of lactobionic acid and 75.44 g of phytosphingosine are dissolved in dimethylformamide (DMF). 2 g of N-hydroxysuccinimide (HSU) and 15 ml of N,N-diisopropylcarbodiimide (DIC) are added and the mixture is reacted at 60° C. for 2 h. Residual DIC is quenched by adding 15 ml of water and allowing the mixture to react for 1/2 hour further. For workup, the dimethylformamide is distilled off and the product residue then dissolved in methanol. The product is then crystallized by cooling slowly to 10° C. The product thus obtained is filtered off, washed with methanol and dried. The yield is >80% and the purity is >90%.

### Example 5: 2-hydroxy-3,3-dimethyl-hydroxybutyroyl phytosphingosine

Phytosphingosine and D-pantolactone are dissolved in ethanol in a 1:1 molar ratio. The mixture is reacted at 60° C. for 8 h. The product is then crystallized by cooling slowly to 10° C. It is then filtered off and dried. The yield is >80% and the purity is >90%.

### Example 5b: 2-hydroxy-3,3-dimethyl-hydroxybutyroyl sphinganine

Sphinganine and D-pantolactone are dissolved in ethanol in a 1:1 molar ratio. The mixture is reacted at 60° C. for 8 h. The product is then crystallized by cooling slowly to 10° C. It is then filtered off and dried. The yield is >80% and the purity is >90%.

Example 6: Prophylaxis against UV Damage

The extent to which the substances of the invention are able to exert a protective effect on normal human epidermal keratinocytes (NHEK) exposed to UV irradiation was examined.

For this, corresponding investigations were carried out of biomarkers for reactive oxygen species (ROS), quantified with 2,7-dichlorodihydrofluorescein diacetate, and DNA damage and repair, quantified by the Comet assay.

Reactive Oxygen Species (ROS)

The intracellular formation of reactive organic species (ROS) is inter alia a factor that leads to damage of cellular DNA by UV radiation.

96-well plates were seeded with keratinocytes, which were cultured for 24 hours in culture medium and then for a further 24 hours in assay medium. The medium was then replaced by a test medium containing the test compounds or the references (100 μM vitamin E and 10 μM EGCG) or in which they were absent (irradiated control) and the cells were preincubated for 24 hours. After preincubation, the medium was removed and replaced by assay medium, the fluorescent probe (2,7-dichlorodihydrofluorescein diacetate (10 μM 2,7-DCDHF-DA in assay medium) was added and the cells were incubated at 37° C. for 30 minutes. The cells were then washed with PBS solution and irradiated with 100 mJ/cm² UVB+UVA (+0.7 J/cm²) without compounds or references. The lamp used was a SOL500 solar simulator with H2 filter (Dr. Honle, AG). After irradiation, the cells were incubated for 30 minutes. An unirradiated control and the conditions without sample (background noise) were carried out in parallel.

All experimental conditions were carried out in triplicate.

The emitted intensity of fluorescence (ex=485 nm, em=538 nm) was measured using an EnVision® (Perkin Elmer) microplate reader.

The intensity of fluorescence of the metabolized sample (DCF) was proportional to the formation of ROS. ROS production was therefore expressed as the relative intensity of fluorescence. FIG. 1 shows the corresponding results.

It can be seen that the production of reactive oxygen species (ROS) in human epidermal keratinocytes is induced by irradiating with UV light (corresponding to 0% protective effect). The vitamin E and epigallocatechin gallate (EGCG) positive controls used in the test are known for having a protective effect against UV irradiation and accordingly show a 30-40% protective effect against the formation of ROS. The employed substances of the invention likewise show with increasing concentration a substantial protective effect of over 40%. Such an effect has not previously been described for sphingolipids/ceramides.

As the base, the sphingoid base phytosphingosine (PS) was also tested. No significant protective effect was discernible here.

The following experiments are not depicted in FIG. 1:
In the above setting 2-hydroxy-3,3-dimethyl-hydroxybutyroyl sphinganine shows a protective effect of 22% at a concentration of 5 μM, 2-hydroxy-3,3-dimethyl-hydroxybutyroyl phytosphingosine shows a protective effect of 38% at a concentration of 5 μM.
In the above setting Ceramide 3 (also known as Ceramide NP) shows a protective effect of 4% at a concentration of 10 μM, 3-hydroxypropioyl phytosphingosine shows a protective effect of 5% at a concentration of 10 μM.

### DNA Damage and Repair

6-well plates were seeded with the keratinocytes, which were incubated for 48 hours in culture medium, with the medium replenished after 24 hours. The medium was then replaced by culture medium containing the test compounds or the references (0.3 mM control) or in which they were absent (control) and the cells were incubated for 24 hours. After the preincubation, the culture medium was removed and assay medium again added and the cells were irradiated with 250 mJ/cm² UVB+UVA (+1.6 J/cm²) in the absence of the compounds. The lamp used was a SOL500 solar simulator (Dr. Hönle, AG) fitted with an H2 filter. An unirradiated control condition was carried out in parallel. All experimental conditions were carried out in duplicate.

At the end of the irradiation, the culture supernatants were discarded and the cells were washed with phosphate-buffered saline (PBS) prior to analysis.

The cells were trypsinized and counted and the supernatant was removed after centrifugation. The cells were then washed with PBS solution and suspended in the same PBS solution so as to achieve a cell concentration of 1×10⁵ cells/ml. The cell suspension was then mixed with molten (37° C.) 1% agarose gel of low melting point and pipetted onto the Comet microscope slides (duplicate analysis for each condition). For cell lysis, the microscope slides were immersed in a freshly prepared alkaline solution (200 mM NaOH containing 1 mM EDTA, pH>13) on an electrophoresis support. The gel electrophoresis was carried out at 21 volts for 30 minutes. The Comet microscope slides were washed twice with water, each time for 5 minutes, then washed with 70% ethanol for 5 minutes and air-dried at 37° C. for 15 minutes.

After the electrophoresis, each dried sample was stained with a DNA intercalating fluorescent dye (SYBR Green solution). The cells were then observed in epifluorescence using an automated IN Cell Analyzer™ 2200 microscope image analyser (GE Healthcare) (X10 objective lens). On excitation (ex 494 nm, em 524 nm), the DNA-bound SYBR® Green emits green light. In healthy cells, the fluorescence is limited to the nucleoid: Undamaged DNA is supercoiled and accordingly does not migrate very substantially from the nucleoid under the influence of an electric current. If DNA damage has occurred, the alkali treatment causes the DNA to roll up, releasing fragments that migrate outside the cell when exposed to an electric field. The negatively charged DNA migrates to the anode, the extrusion length being proportional to the relaxation of the supercoiled structure, which is an indicator of damage. When alkaline electrophoresis conditions are employed, the distribution of the DNA between the tail and comet head can be used to determine the extent of the DNA damage. The images of the cells were analysed with OpenComet in conjunction with ImageJ software. In each replicate of the analysis, a minimum value of 500 events was analysed.

FIG. 2 shows the corresponding results.

DNA damage caused by the irradiation of the human epidermal keratinocytes with UV light is discernible. The tiron positive control is able, by virtue of its known antioxidant properties, to protect against DNA damage and accordingly shows an approximately 60% protective effect against DNA damage after UV irradiation. The substances of the invention likewise show a protective effect of more than 60%. This effect is not known for sphingolipids/ceramides and also cannot be demonstrated for the pure sphingoid base phytosphingosine (PS) in this test.

The following experiments are not depicted in FIG. 2:
In the above setting 2-hydroxy-3,3-dimethyl-hydroxybutyroyl sphinganine shows a protective effect of 24% at a concentration of 5 μM, 2-hydroxy-3,3-dimethyl-hydroxybutyroyl phytosphingosine shows a protective effect of 39 % at a concentration of 2 μM.
In the above setting Ceramide 3 (also known as Ceramide NP) shows a protective effect of 12% at a concentration of 10 μM, 3-hydroxypropioyl phytosphingosine shows a protective effect of 13% at a concentration of 10 μM.

### Example 7: In-Vivo Data

For the in-vivo study, 24 test subjects (male and female) with sun-stressed skin were recruited. In order to ensure sun-stressed skin, this study was carried out in the period from September to November. The assumption was that skin shows the greatest level of sun stress at the end of the summer season.

The test subjects received either two different test formulations, which they applied to one forearm each, or one test formulation. which was applied to one forearm with the second forearm remaining untreated (control). The test formulations were a vehicle and a formulation containing 0.1% of hydroxybutyroyl phytosphingosine (hydroxybutyroyl PS). The various test combinations were assigned to the test subjects randomly.

The composition of the test formulation is shown in Table 1. The test subjects applied the test formulations for a period of 8 weeks twice daily to the inside and outside of in each case one forearm. The following measurements on the forearms were carried out before the start of application and after two, four and eight weeks:
1. Colour measurement: A colorimeter probe (Skin-Colorimeter CL 400, Courage & Khazaka, Cologne) was used to measure the parameters L* and ITA° on the outside of the forearm. L* describes the black/white value of the skin, ITA° describes the skin tone. When skin becomes lighter, both values show an increase.
2. Skin roughness: This parameter was determined by means of a special camera on the inside of the forearm (Visioscan VC 98, Courage & Khazaka). This camera records a digital black-and-white image of the skin. The grayscale distribution of the image can be used to determine the skin roughness.
3. Density of the dermis: The density of the dermis was determined by ultrasound on the outside of the forearm (SkinLab Combo, Cortex Technologies, Denmark). For this, a special probe transmits an ultrasound signal to the skin and records the reflection. This reflection can be used to determine a value for the skin density. A reduced skin density is shown in particular by areas of skin that have been very strongly exposed to sunlight.

TABLE 1
Composition of the test formulations

| | | | Hydroxybutyroyl |
|---|---|---|---|
| | | Vehicle | phytosphingosine |
| Phase | Ingredient | [% w/w] | [% w/w] |
| A | TEGO ® Care 450 | 3.00 | 3.00 |
| | (Polyglyceryl-3 | | |
| | Methylglucose Distearate) | | |
| | TEGIN ® M Pellets | 2.00 | 2.00 |
| | (Glyceryl Stearate) | | |
| | TEGO ® Alkanol 1618 | 1.00 | 1.00 |
| | (Cetearyl Alcohol) | | |
| | TEGOSOFT ® CT (Caprylic/ | 9.50 | 9.50 |
| | Capric Triglyceride) | | |
| | TEGOSOFT ® TN | 9.50 | 9.50 |
| | (C12-15 Alkyl Benzoate) | | |
| B | Hydroxybutyroyl | | 0.10 |
| | phytosphingosine | | |
| | Water | 71.00 | 70.90 |
| | Glycerol | 3.00 | 3.00 |
| Z | Verstatil PC (Phenoxyethanol; | 1.00 | 1.00 |
| | Caprylyl Glycol) | | |

FIG. 3 shows the increase in ITA° after application of the test formulations for two, four and eight weeks.

FIG. 4 shows the increase in L* after application of the test formulations for two, four and eight weeks.

The increase in the colour parameters L* and ITA° in Figures xy1 and xy2 shows that the skin became brighter over the whole measurement period, the increase in brightness being most pronounced, compared with the vehicle formulation or the untreated control, on the areas of skin treated with hydroxybutyroyl phytosphingosine. This was in this case not attributable to destruction of melanin in the skin. Tanning of the skin is most pronounced at the end of the summer season and then subsides in autumn as a consequence of the normal skin regeneration cycle. This cycle is intensified by the hydroxybutyroyl phytosphingosine, which in this case results in more rapid lightening of the skin.

FIG. 5 shows the decrease in skin roughness after application of the test formulations for two, four and eight weeks.

From FIG. 5 it can be seen that the decrease in skin roughness was most pronounced with the test formulation containing hydroxybutyroyl phytosphingosine, both in comparison to the untreated control and particularly in comparison with the vehicle. This supports the skin colour measurement results: a characteristic feature of sun-stressed skin is that the roughness of the skin is increased. This roughness likewise decreases towards autumn as a consequence of normal skin regeneration. Because hydroxybutyroyl phytosphingosine aids the skin renewal cycle, the decrease in skin roughness is more pronounced than with the vehicle formulation or the untreated control.

FIG. 6 shows the increase in skin density after application of the test formulations for two, four and eight weeks.

Areas of skin with greater exposure to solar radiation show a reduced skin density. The results of the skin study show that the skin density has with hydroxybutyroyl phytosphingosine already increased markedly after two weeks of use compared with the untreated control or the vehicle.

The experiments are repeated in an identical way with 6-hydroxyhexanoyl phytosphingosine (example 1b), 2-hydroxy-3,3-dimethyl-hydroxybutyroyl sphinganine (example 5b), Ceramide 3 (also known as Ceramide NP) and 3-hydroxypropioyl phytosphingosine (example 1c)

| | ΔITA° | | | ΔL* | | | Decrease in roughness | | | Δ skin density | | |
|---|---|---|---|---|---|---|---|---|---|---|---|---|
| | W2 − W0 | W4 − W0 | W8 − W0 | W2 − W0 | W4 − W0 | W8 − W0 | W2 − W0 | W4 − W0 | W8 − W0 | W2 − W0 | W4 − W0 | W8 − W0 |
| Ex 1b | 5.11 | 7.53 | 12.24 | 1.38 | 2.51 | 4.38 | −6.20 | −14.36 | −15.88 | 3.33 | 4.24 | 4.63 |
| Ex 5b | 3.54 | 6.21 | 10.52 | 0.92 | 2.02 | 3.28 | −3.2 | −12.34 | −13.63 | 2.23 | 3.25 | 3.64 |
| Cer 3 | 2.34 | 5.21 | 8.79 | 0.60 | 1.82 | 2.61 | 0.05 | −5.12 | −4.66 | 1.20 | 1.50 | 1.80 |
| Ex 1c | 2.41 | 4.89 | 8.67 | 0.58 | 1.92 | 2.55 | −1.20 | −5.55 | −6.21 | .82 | 0.98 | 0.82 |

1. A sphingolipid of the general formula I 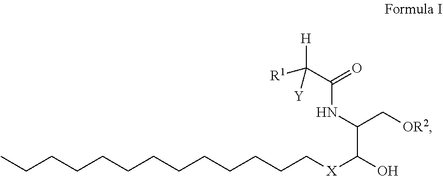
wherein
R¹ is a hydrocarbon radical having 2 to 54 carbon atoms that is substituted with at least one group selected from the group consisting of —OH and —COOH, and that optionally may be interrupted by at least one —O—,
R² is H, phosphocholine, serine, ethanolamine, or a sugar,
X is CH═CH, CH₂-CH₂, or CH₂-HCOH, and
Y is selected from the group consisting of —OH and H,
with the proviso that, when R¹ is a linear alkyl radical substituted exclusively with an —OH group in the ω-position, X is CH₂-HCOH.

2. The sphingolipid according to claim 1, wherein
R¹ is a linear or branched alkyl radical having 2 to 54 carbon atoms that is substituted with at least one group selected from the group consisting of —OH and —COOH, and
R², Y are H, and
X is CH₂-HCOH.

3. The sphingolipid according to claim 1, wherein
R¹ is a hydrocarbon radical haying 2 to 54, carbon atoms that is substituted at the ω-position with at least one group selected from the group consisting of —OH and —COOH, and
R², Y are H, and
X is CH₂-HCOH,

4. The sphingplipid according to claim 1, selected from the group consisting of
hydroxybutyroyl phytosphingosine 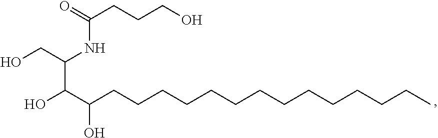 [SMILES: CCCCCCCCCCCCCCC(O)C(O)C(CO)NC(=O)CCCO]
succinoyl phytosphingosine 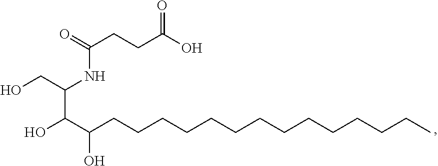 [SMILES: CCCCCCCCCCCCCCC(O)C(O)C(CO)NC(=O)CCC(=O)O]
gluconoyl phytosphingosine 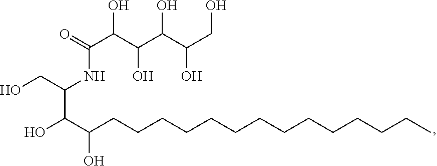 [SMILES: CCCCCCCCCCCCCCC(O)C(O)C(CO)NC(=O)C(O)C(O)C(O)C(O)CO]
lactobionoyl phytosphingosine 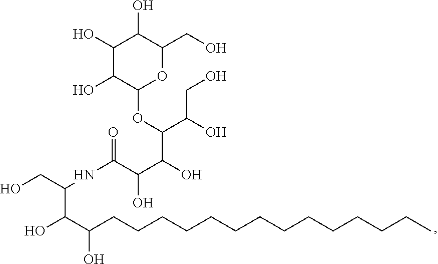 [SMILES: CCCCCCCCCCCCCCC(O)C(O)C(CO)NC(=O)C(O)C(O)C(OC1OC(CO)C(O)C(O)C1O)C(O)CO]
and
2-hydroxy-3,3-dimethyl-hydroxybutyroyl phytosphingosine

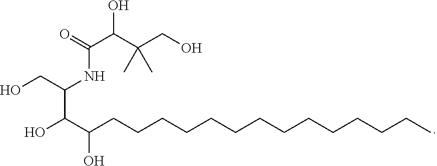 [SMILES: CCCCCCCCCCCCCCC(O)C(O)C(CO)NC(=O)C(O)C(C)(C)CO]

5. A method for therapy, or prophylaxis of cell damage induced by UV radiation, or skin cell damage, the method comprising:
applying the sphingolipid according to claim 1 to skin.

6. A method for the prevention of skin ageing caused by UV radiation, the method comprising:
applying the sphingolipid accordin2 to claim 1 to skin.

7. A cosmetic formulation, comprising:
at least one sphingolipid according to claim 1.

8. The cosmetic formulation according to claim 7, additionally comprising:
at least one UV light protection filter substance.

9. A method, for preparing the sphingolipid according to claim 1, the method comprising:
I) providing as a first component, at least a lysosphingolipid of the general formula II 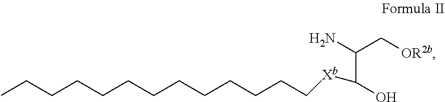
wherein
R^{2b} is phosphocholine, serine, ethanolamine, or a sugar, and
X^{b} is CH═CH, CH₂-CH₂, or CH₂-HCOH, and
II) providing as a second component, at least an intramolecular cyclic ester of a hydroxycarboxylic acid of the formula R^{1b}CY^{b}HCOOH,
wherein
R^{1b} is a hydrocarbon radical having 2 to 54, carbon atoms that is substituted with at least one group selected from the group consisting of —OH and —COOH, and that optionally may be interrupted by at least one —O—, and
Y^{b} is selected from the group consisting of —OH and H,
III) reacting the first component with the second component to obtain the sphingolipid, and
IV) optionally, purifying the sphingolipid.

10. The method according to claim 9, wherein III) is carried out within a temperature range from 40° C. to 95° C.

11. A method for preparing the sphingolipid accordine to claim 1,. the method comprising:
A) providing as a first component, at least a lysosphingolipid of the general formula II 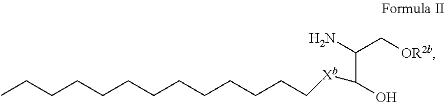
wherein
R²b is H, phosphocholine, serine, ethanolamine, or a sugar, and
X^{b} is CH═CH, CH₂-CH₂, or CH₂-HCOH, and
B) providing as a second component, at least a hydroxycarboxylic acid of the formula R^{1b}CY^{b}HCOOH,
wherein
R^{1b} is a hydrocarbon radical having 2 to 54 carbon atoms that is substituted with at least one group selected from the group consisting of —OH and —COOH and that optionally Wray be interrupted by at least one —O—,
Y^{b} is selected from the group consisting of —OH and H, and
C) reacting the first component with the second component, with at least one coupling reagent for activation of the hydroxycarboxylic acid, to obtain the sphingolipid, and
D) optionally, purifying the sphingolipid.

12. The method according to claim 11, wherein in C) the at least one coupling reagent is at least one selected from the group consisting of dicyclohexylcarbodiimide, diisopropylcarbodiimide, 1-(3-dimethylaminopropyl)-3-ethylcarbodiimide hydrochloride, N-cyclohexyl-N′-(2′-morpholinoethyl)carbodiimide metho-p-toluenestilfonate, N-benzyl-N′-3′dimethylaminopropylcardiimide hydrochloride, 1-ethyl-3-(3-dimethylaminopropyl)carbodiimide, N-ethylcarbodiimide hydrochloride, and carbonyldiimidazole.

13. The method according to claim 11, wherein the reacting C) is with at least one catalyst selected from the group comprising consisting of N-ethyldiisopropylamine, trilkyamines, pyridine, 4-dimethylaminopyridine, and hydroxybenzotriazole.

14. The sphingolipid according to claim 1, wherein
R¹ is a hydrocarbon radical having 2 to 18 carbon atoms that is substituted with at least one group selected from the group consisting of —OH and —COOH, and that is optionally interrupted by at least one —O—.

15. The sphingolipid according to claim 1, wherein
R² is a sugar or H.)

16. The cosmetic formulation according to claim 7, comprising the at least one sphingolipid in an amount of 0.02% by weight to 1.50% by weight, based on the total cosmetic formulation.

17. The cosmetic formulation according to claim 7, comprising the at least one sphingolipid in an amount of 0.05% by weight to 0.50% by weight, based on the total cosmetic formulation.

18. The method according to claim 10, wherein the temperature range is from 60° C. to 70° C.

19. The method according to claim 12, wherein the at least one coupling reagent is dicyclohexylcarbodiimide or diisopropylcarbodiimide.

20. The method according to claim 13, wherein the at least one catalyst is hydroxybenzotriazole.
